(19) United States
(12) Patent Application Publication
(10) Pub. No.: US 2019/0006024 A1
(43) Pub. Date: Jan. 3, 2019

(54) METHODS AND SYSTEMS FOR MATCHING PATIENTS WITH CLINICAL TRIALS

(71) Applicant: Trishul Kapoor, San Jose, CA (US)

(72) Inventor: Trishul Kapoor, San Jose, CA (US)

(21) Appl. No.: 15/853,925

(22) Filed: Dec. 25, 2017

Related U.S. Application Data
(60) Provisional application No. 62/438,465, filed on Dec. 23, 2016.

(51) Int. Cl. G16H 10/20; G16H 10/60

(52) U.S. Cl. CPC G16H 10/20; G16H 10/60

(57) ABSTRACT

In one aspect, a computerized method useful for matching patients with clinical trials includes the step of obtaining a patient Electronic medical record (EMR) data. The computerized method includes the step of parsing the patient EMR data into a set of patient attributes. The computerized method includes the step of obtaining a set of clinical-trial parameters for a clinical trial. The computerized method includes the step of matching at least one patient attribute to the set of clinical-trial parameters. The computerized method includes the step of ranking a set of matched patients based on a strength value of the matching at least one patient attribute to the set of clinical-trial parameters.

### CROSS-REFERENCE TO RELATED APPLICATIONS

This application is a claims priority to U.S. provisional patent application No. 62/438,465, titled METHODS AND SYSTEMS FOR MATCHING PATIENTS WITH CLINICAL TRIALS and filed on Dec. 23, 2016. This application is hereby incorporated by reference in their entirety.

### BACKGROUND

Electronic medical record (EMR) data is available in digital formats. Clinical trials may benefit from the use of EMR data. For example, clinical trials may seek specific ranges/types of patients. It may be difficult to obtain this information from current sources. Digital EMR data can provide faster and more accurate patient information.

### BRIEF SUMMARY OF THE INVENTION

In one aspect, a computerized method useful for matching patients with clinical trials includes the step of obtaining a patient Electronic medical record (EMR) data. The computerized method includes the step of parsing the patient EMR data into a set of patient attributes. The computerized method includes the step of obtaining a set of clinical-trial parameters for a clinical trial. The computerized method includes the step of matching at least one patient attribute to the set of clinical-trial parameters. The computerized method includes the step of ranking a set of matched patients based on a strength value of the matching at least one patient attribute to the set of clinical-trial parameters. The computerized method includes the step of enabling the set of matched patients to enroll in the clinical trial.

### BRIEF DESCRIPTION OF THE DRAWINGS

FIG. 1 illustrates an example system utilized matching patients with clinical trials, according to some embodiments.

FIG. 2 depicts an exemplary computing system that can be configured to perform any one of the processes provided herein.

FIG. 3 is a block diagram of a sample-computing environment that can be utilized to implement various embodiments.

FIG. 4 illustrates an example dashboard for a medical provider in a system utilized for matching patients with clinical trials, in according to some embodiments.

FIGS. 5-6 illustrates an example set of dashboards for a patient in a system utilized for matching patients with clinical trials, in according to some embodiments.

FIG. 7 illustrates an example process of matching patients with clinical trials, according to some embodiments.

The Figures described above are a representative set, and are not an exhaustive with respect to embodying the invention.

### DESCRIPTION

Disclosed are a system, method, and article for matching patients with clinical trials. The following description is presented to enable a person of ordinary skill in the art to make and use the various embodiments. Descriptions of specific devices, techniques, and applications are provided only as examples. Various modifications to the examples described herein can be readily apparent to those of ordinary skill in the art, and the general principles defined herein may be applied to other examples and applications without departing from the spirit and scope of the various embodiments.

Reference throughout this specification to “one embodiment,” “an embodiment,” ‘one example,’ or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, and they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

### Definitions

Example definitions for some embodiments are now provided.

Clinical trials can be experiments done in clinical research (e.g. medical clinical trials, etc.). Prospective biomedical and/or behavioral research studies on human participants can be designed to answer specific questions about biomedical or behavioral interventions, including new treatments (e.g. novel vaccines, drugs, dietary choices, dietary supplements, medical devices, etc.).

Cloud computing can involve deploying groups of remote servers and/or software networks that allow centralized data storage and online access to computer services or resources. These groups of remote serves and/or software networks can be a collection of remote computing services.

Electronic medical record (EMR) is a medical record in digital format.

Mobile device can be a smart phone, tablet computer, wearable computer (e.g. a smart watch, a head-mounted display computing system, etc.). In one example, a mobile device can be a small computing device, typically small enough to be handheld having a display screen with touch input and/or a miniature keyboard.

Ranking is a relationship between a set of items such that, for any two items, the first is either ‘ranked higher than’, ‘ranked lower than’ or ‘ranked equal to’ the second.

Recommendation systems can be a subclass of information filtering system that predicts a rating and/or preference.

Exemplary Systems

In some embodiments, a system for matching patients with clinical trials. Clinical trials may seek to study specific medical issues. Clinical trials may seek patients (and/or other types of users in other examples) with specified characteristics/qualities. Said patient characteristics can include, inter alia: demographic backgrounds, age, disease history, genetic profiles, family health history, location, lifestyle choices, drug use, physiological characteristics, medical treatment histories, exercise history, physician's office location, past-clinical trial history, etc. Clinical trials can be performed by various entities such as, inter alia: pharmaceutical companies, research organizations, universities, etc.

Various entities can log into a system for matching patients with clinical trials via specialized dashboards. For example, medical care providers can be provided a specialized dashboard. Patients can be provided a specialized dashboard. Clinical trial providers can be provider a specialized dashboard. These dashboards can enable entities to provide various permissions, attributes, requests, etc. that enable the entities to be matched. Matches can be based on matching requested patient attributes by upload patient attributes by patients and/or medical care providers. It is noted that in some embodiments, local EMR data stores of a medical care provider can be utilized. The system for matching patients with clinical trials can manage Health Insurance Portability and Accountability Act (HIPPA)-related and/or other privacy/legal issues related to sharing patient medical data. The system for matching patients with clinical trials can anonymize portions of patient medical data.

The system for matching patients with clinical trials can maintain a patient rating system. The rating system can be based on, for example, surveys completed by patients, trials completed by patients, money earned by patients, various matching system information, location based detection for clinical trials, etc. The system for matching patients with clinical trials can enable a function that enables mobile device tracking of patients during specified periods and/or other methods of verify patient behavior during a clinical trial. The system for matching patients with clinical trials can push patient's mobile device questions for surveys, requests for EMR data permissions, etc. The system for matching patients with clinical trials can link patients to specific clinical trials that match the patient's medical needs.

EMR systems can be the systematized collection of patient and population electronically-stored health information in a digital format. These records can be shared across different health care settings. Records can be shared through network-connected, enterprise-wide information systems or other information networks and exchanges. EMR can include a range of data, including, inter alia: demographics, medical history, medication and allergies, immunization status, laboratory test results, radiology images, vital signs, personal statistics like age and weight, and billing information, etc.

EMR systems can be designed to store data accurately and to capture the state of a patient across time. EMR systems can eliminate the need to track down a patient's previous paper medical records and assist in ensuring data is accurate and legible. Due to the digital information being searchable and in a single file, EMR systems can be more effective when extracting medical data for the examination of possible trends and long-term changes in a patient. Population-based studies of medical records may also be facilitated by the widespread adoption of EMR systems.

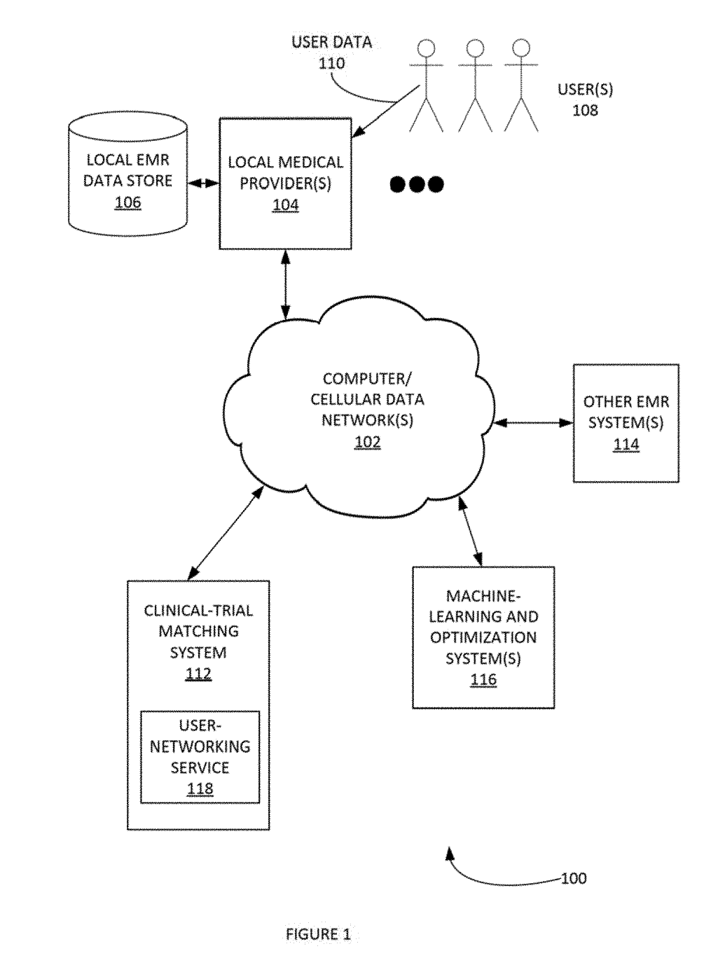

FIG. 1 illustrates an example system 100 utilized matching patients with clinical trials, according to some embodiments. System 100 can include various computerized entities that communicated via computer and/or cellular data networks 102 (e.g. the Internet, etc.). Local medical providers 104 can store user data 110 to a local EMR data store 106. Users 108 can provide various permissions for the use of user data 110 via local medical provider 104. Additionally, users 108 can access clinical-trial matching system 112 and manage the uploading of the user's medical data (e.g. via dashboard of a web page, etc.).

Clinical-trial matching system 112 can receive user medical data and/or other user-related information from local medical providers 104, other EMR systems 114 and/or users 108. Other EMR systems 114 can include a non-local EMR. An example other EMR systems 114 can be associated with an insurance provider, a health-care entity, a hospital, etc. Clinical-trial matching system 112 can receive clinical-trial information (e.g. from a university, a clinical-care entity, a pharmaceutical company, a non-profit organization, a governmental organization, a law enforcement organization, etc.). Clinical-trial matching system 112 can include various matching functionalities to match user medical data and/or other user-related information with relevant clinical-trial information. Clinical-trial matching system 112 can include various machine-learning engines, optimization engines, recommendation engines, ranking engines, and the like, in order to optimize the matching process. These optimize the matching process can be located in machine-learning and optimization system(s) 116. The various entities of system 100 can be implemented in servers and/or cloud-computing platforms. Clinical-trial matching system 112 can electronically communicate (e.g. via dashboard notifications, email, text message, etc.) with local medical providers 104 to confirm patient data. Clinical-trial matching system 112 can include various functionalities such as, inter alia: web servers, recommendation engines, databases, database management systems, e-mail servers, statistics engines, etc. Clinical-trial matching system 112 can implement process 700 discuss infra.

Ranking engines can implement sorting algorithms based on the various patient attributes, etc. discussed herein. a sorting algorithm is an algorithm that puts elements of a list in a certain order. Example orders can be numerical order and lexicographical order. The output can satisfy two conditions: the output is in nondecreasing order (each element is no smaller than the previous element according to the desired total order); the output is a permutation (reordering but with the original elements) of the input. Example sorting algorithms can include, inter alia: classification algorithms, quicksort, merge sort, In-place merge sort, heapsort, insertion sort, introsort, selection sort, time sort, cube sort, binary tree sort, smooth sort, cycle sort, etc.

Clinical-trial matching system 112 can include a user-networking platform 118 that enables various users to communicate and exchange data. For example, user-networking platform 118 can include an online social media platform and social networking service. This online social media platform and social networking service can be specialized for the exchange of clinical-trial information. In this way, user-networking service 118 can provide a social marketplace for the clinical-trial information of clinical-trial matching system 112. User-networking platform 118 can communicate between the entities of clinical-trial matching system 112 and various other medical-service providers, health organizations, patients, pharmaceutical companies and/or other medical service companies (e.g. biomedical device companies, prosthetic companies, governmental health entities, etc.).

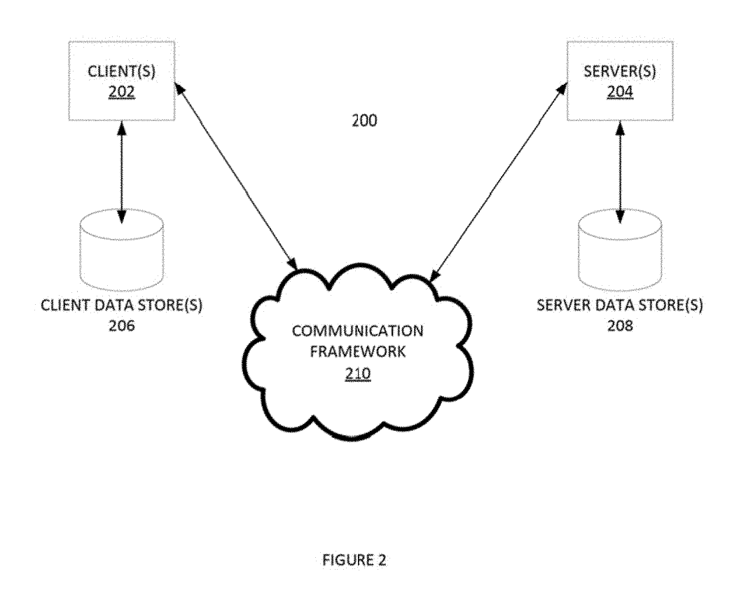

FIG. 2 is a block diagram of a sample-computing environment 200 that can be utilized to implement various embodiments. The system 200 further illustrates a system that includes one or more client(s) 202. The client(s) 202 can be hardware and/or software (e.g., threads, processes, computing devices). The system 200 also includes one or more server(s) 204. The server(s) 204 can also be hardware and/or software (e.g., threads, processes, computing devices). One possible communication between a client 202 and a server 204 may be in the form of a data packet adapted to be transmitted between two or more computer processes. The system 200 includes a communication framework 210 that can be employed to facilitate communications between the client(s) 202 and the server(s) 204. The client(s) 202 are connected to one or more client data store(s) 206 that can be employed to store information local to the client(s) 202. Similarly, the server(s) 204 are connected to one or more server data store(s) 208 that can be employed to store information local to the server(s) 204. In some embodiments, system 200 can instead be a collection of remote computing services constituting a cloud-computing platform.

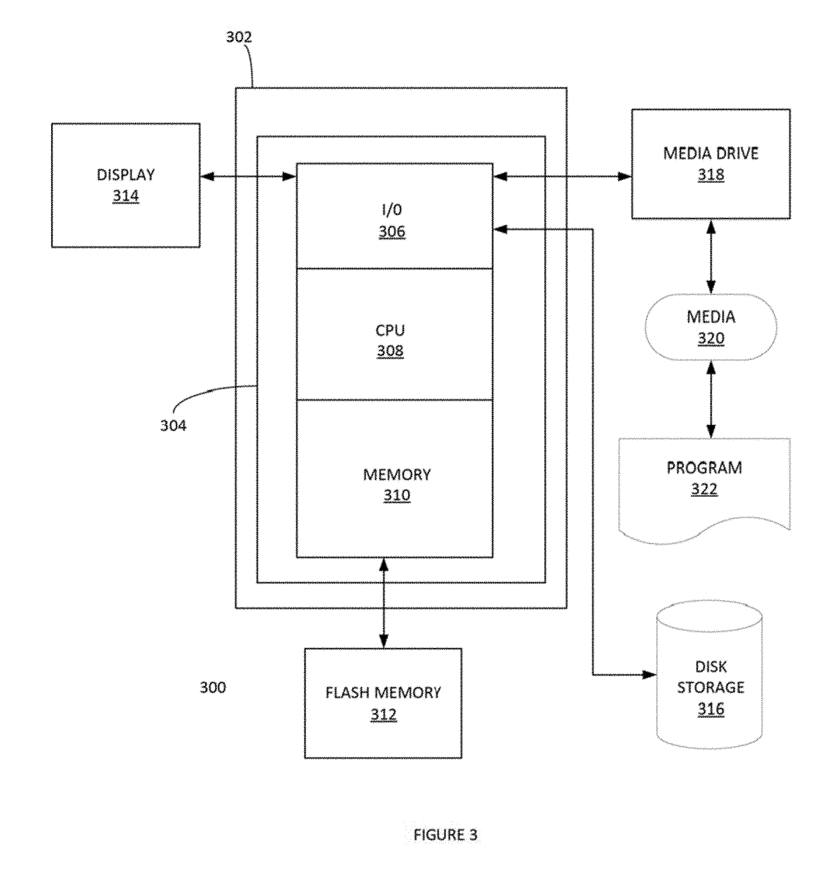

FIG. 3 depicts an exemplary computing system 300 that can be configured to perform any one of the processes provided herein. In this context, computing system 300 may include, for example, a processor, memory, storage, and I/O devices (e.g., monitor, keyboard, disk drive, Internet connection, etc.). However, computing system 300 may include circuitry or other specialized hardware for carrying out some or all aspects of the processes. In some operational settings, computing system 300 may be configured as a system that includes one or more units, each of which is configured to carry out some aspects of the processes either in software, hardware, or some combination thereof.

FIG. 3 depicts computing system 300 with a number of components that may be used to perform any of the processes described herein. The main system 302 includes a motherboard 304 having an I/O section 306, one or more central processing units (CPU) 308, and a memory section 310, which may have a flash memory card related to it. The I/O section 306 can be connected to a display 314, a keyboard and/or other user input (not shown), a disk storage unit 316, and a media drive unit 318. The media drive unit 318 can read/write a computer-readable medium 320, which can contain programs 322 and/or data. Computing system 300 can include a web browser. Moreover, it is noted that computing system 300 can be configured to include additional systems in order to fulfill various functionalities. Computing system 300 can communicate with other computing devices based on various computer communication protocols such a Wi-Fi, Bluetooth® (and/or other standards for exchanging data over short distances includes those using short-wavelength radio transmissions), USB, Ethernet, cellular, an ultrasonic local area communication protocol, etc.

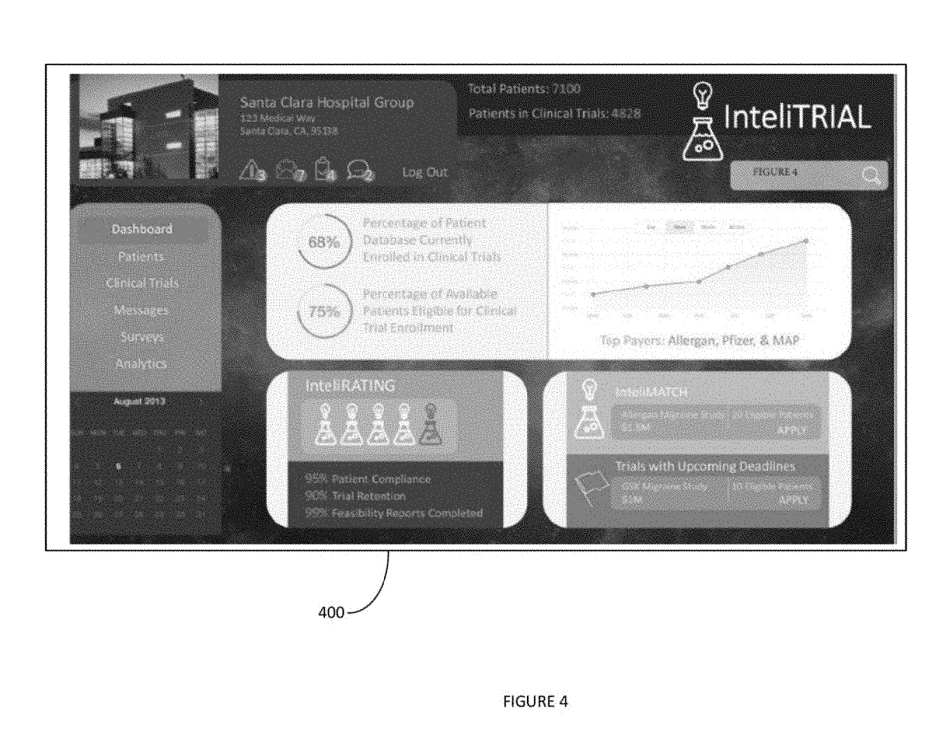

FIG. 4 illustrates an example dashboard for a medical provider in a system utilized for matching patients with clinical trials, in according to some embodiments. Dashboard for a medical provider can show various information related clinical trials and/or the medical provider's patient population. Dashboard for a medical provider can show a percentage of patients that are currently enrolled in clinical trials. Dashboard for a medical provider can show a percent of patients that are available for clinical trials. Dashboard for a medical provider can show the privacy/permission status of patients. Dashboard for a medical provider can show matches between patients and clinical trials. Dashboard for a medical provider can enable the medical provider to recommend patients for specific clinical trials. Dashboard for a medical provider can show one or more criteria that the medical provider has selected (e.g. whether doctor wants reimbursement, specified conditions, specified patients to nominate, etc.). In this way, a medical provide can focus on certain types of clinical trials (e.g. dermatology, neurology, surgery, pediatrics, dental, etc.). Dashboard for a medical provider can provide a summary of patient activity, patient earnings, medical provider earnings, communications with patients, etc.

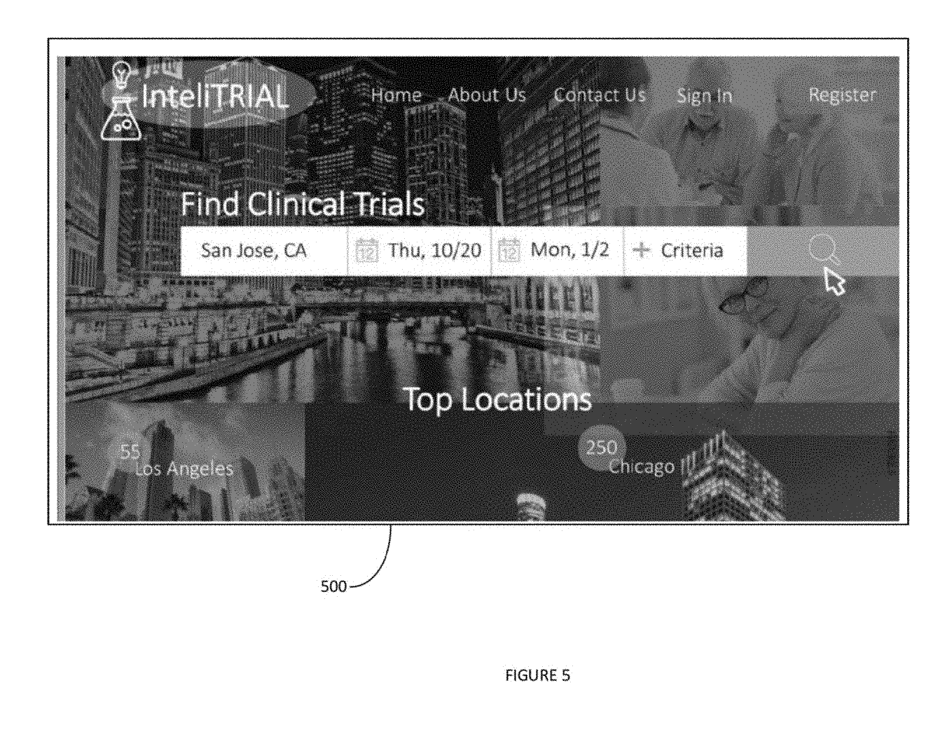

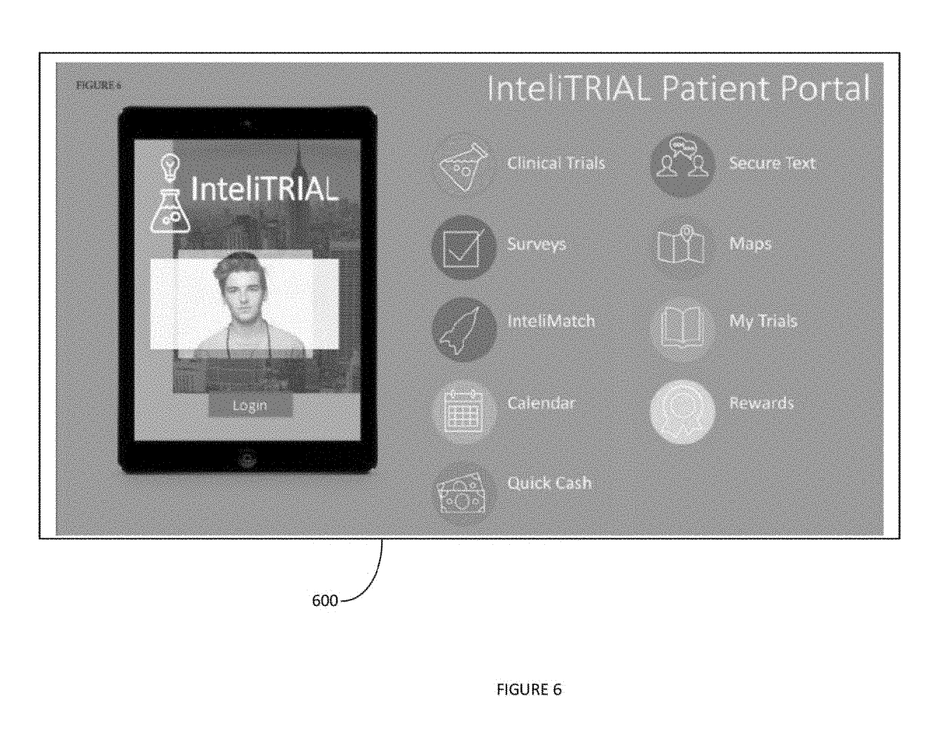

FIGS. 5-6 illustrates an example dashboards for a patient in a system utilized for matching patients with clinical trials, in according to some embodiments. Dashboard for a patient provide a patient portal. For example, a patient can have a personalize dashboard. A patient's dashboard can be available on smartphone, website, etc. A patient dashboard can provide a summary of patient activity, patient earnings, patient communications with doctor.

### Example Methods and Use Cases

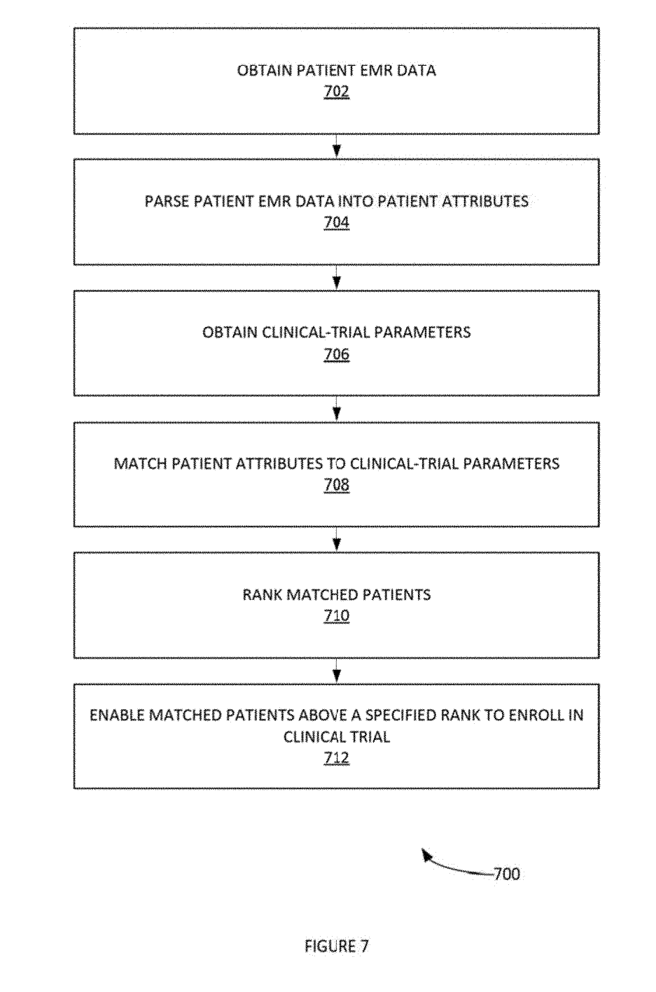

FIG. 7 illustrates an example process 700 of matching patients with clinical trials, according to some embodiments. In step 702, process 700 can obtain patient EMR data. In step 704, process 700 parse patient EMR data into patient attributes. In step 706, process 700 can obtain clinical-trial parameters. Example clinical-trial parameters can include pharmaceutical regimes, exercise regimes, other medical procedures, location of clinical trial events, length of clinical trial, attributes of participants/patients, entities to administer clinical trial protocols, disqualification parameters (e.g. presence of a patient-attribute parameter, demographic attribute, age, etc. In step 708, process 700 can match patient attributes to clinical-trial parameters. Patient attributes can include, inter alia: including, inter alia: demographics, medical history, medication and allergies, clinical-trial participation history, immunization status, laboratory test results, radiology images, vital signs, personal statistics like age and weight, and billing information. In step 710, process 700 can rank matched patients. In step 712, process 700 can enable matched patients above a specified rank to enroll in clinical trial. Process 700 can match a patient to a clinical trial through various other factors, such as: doctor identity, doctor location, clinical-trial location(s), etc.

### CONCLUSION

Although the present embodiments have been described with reference to specific example embodiments, various modifications and changes can be made to these embodiments without departing from the broader spirit and scope of the various embodiments. For example, the various devices, modules, etc. described herein can be enabled and operated using hardware circuitry, firmware, software or any combination of hardware, firmware, and software (e.g., embodied in a machine-readable medium).

In addition, it can be appreciated that the various operations, processes, and methods disclosed herein can be embodied in a machine-readable medium and/or a machine accessible medium compatible with a data processing system (e.g., a computer system), and can be performed in any order (e.g., including using means for achieving the various operations). Accordingly, the specification and drawings are to be regarded in an illustrative rather than a restrictive sense. In some embodiments, the machine-readable medium can be a non-transitory form of machine-readable medium.

What is claimed as new and desired to be protected by Letters Patent of the United States is:

1. A computerized method useful for matching patients with clinical trials comprising:
obtaining a patient Electronic medical record (EMR) data;
parsing the patient EMR data into a set of patient attributes;
obtaining a set of clinical-trial parameters for a clinical trial;
matching at least one patient attribute to the set of clinical-trial parameters;
ranking a set of matched patients based on a strength value of the matching at least one patient attribute to the set of clinical-trial parameters; and
enabling the set of matched patients to enroll in the clinical trial.

2. The computerized method of claim 1, wherein the set of clinical-trial parameters comprises a pharmaceutical regime.

3. The computerized method of claim 2, wherein the set of clinical-trial parameters comprises an exercise regime, a location of the clinical trial, and a length of clinical trial.

4. The computerized method of claim 3, wherein the set of clinical-trial parameters comprises an attribute of a participant and an identity of an entity to administer clinical trial protocols.

5. The computerized method of claim 4, wherein the set of clinical-trial parameters comprises a disqualification parameter.

6. The computerized method of claim 5, wherein the least one patient attribute comprises a demographic attribute, a medical history attribute, and a medication allergy attribute.

7. The computerized method of claim 6, wherein the least one patient attribute comprises a clinical-trial participation history attribute, an immunization status attribute, and a laboratory test result attribute.

8. The computerized method of claim 7, wherein the least one patient attribute comprises a radiology image attribute, a vital sign attribute, a personal statistic attribute.

9. The computerized method of claim 8 wherein the personal statistic attribute comprises a patient age, a patient weight, and a billing information.

10. The computerized method of claim 9, wherein is based on a set of requested patient attributes provided by a medical care provider.

11. The computerized method of claim 10, wherein the patient is matched to a clinical trial based on a doctor identity, a doctor location, and a clinical-trial location.

12. A computer system useful for matching patients with clinical trials comprising:
at least one processor configured to execute instructions;
a memory containing instructions when executed on the processor, causes the at least one processor to perform operations that:
obtain a patient Electronic medical record (EMR) data;
parse the patient EMR data into a set of patient attributes;
obtain a set of clinical-trial parameters for a clinical trial;
match at least one patient attribute to the set of clinical-trial parameters;
rank a set of matched patients based on a strength value of the matching at least one patient attribute to the set of clinical-trial parameters; and
enable the set of matched patients to enroll in the clinical trial.

13. The computerized system of claim 12, wherein the set of clinical-trial parameters comprises a pharmaceutical regime.

14. The computerized system of claim 13, wherein the set of clinical-trial parameters comprises an exercise regime, a location of the clinical trial, and a length of clinical trial.

15. The computerized system of claim 14, wherein the set of clinical-trial parameters comprises an attribute of a participant and an identity of an entity to administer clinical trial protocols.

16. The computerized system of claim 15, wherein the set of clinical-trial parameters comprises a disqualification parameter.

17. The computerized system of claim 16, wherein the least one patient attribute comprises a demographic attribute, a medical history attribute, and a medication allergy attribute.

18. The computerized system of claim 17, wherein the least one patient attribute comprises a radiology image attribute, a vital sign attribute, a personal statistic attribute.

19. The computerized system of claim 18, wherein is based on a set of requested patient attributes provided by a medical care provider.

20. The computerized system of claim 19, wherein the patient is matched to a clinical trial based on a doctor identity, a doctor location, and a clinical-trial location.
